(19) United States
(12) Patent Application Publication
(10) Pub. No.: US 2010/0216848 A1
(43) Pub. Date: Aug. 26, 2010

(54) LIQUID PHARMACEUTICAL COMPOSITIONS FOR PARENTERAL ADMINISTRATION OF A SUBSTITUTED AMIDE

(76) Inventors: Andrey V. Peresypkin, Harleysville, PA (US); Sachin Mittal, Telford, PA (US)

Correspondence Address: MERCK, P O BOX 2000, RAHWAY, NJ, 07065-0907, US

(21) Appl. No.: 12/678,783

(22) PCT Filed: Oct. 6, 2008
(86) PCT No.: PCT/US08/11516

§ 371 Date: Mar. 18, 2010

Related U.S. Application Data
(60) Provisional application No. 60/998,428, filed on Oct. 10, 2007.

(51) Int. Cl. A61K 31/4412; A61P 3/10; A61P 3/04

(52) U.S. Cl. 514/351

(57) ABSTRACT

A liquid pharmaceutical composition comprising N-[1S,2S]-3-[(4-chlorophenyl)-2-(3-cyanophenyl)-1-methylpropyl]-2-methyl-2-{[(5-trifluoromethyl)pyridine-2-yl]oxy}propanamide is disclosed, together with processes of preparing the liquid pharmaceutical composition, preparing a vessel containing the liquid pharmaceutical composition, the use of the composition for treating and preventing diabetes or obesity and a method of treating or preventing diabetes or obesity comprising administration to a patient in need thereof of an effective amount of the liquid pharmaceutical composition.

### BACKGROUND OF THE INVENTION

N-[1S,2S]-3-[(4-chlorophenyl)-2-(3-cyanophenyl)-1-methylpropyl]-2-methyl-2-{[(5-trifluoromethyl)pyridine-2-yl]oxy}propanamide (Compound I), described in WO 03/077847, is a cannabinoid 1 (CB1) receptor modulator, more particularly a functional CB1 antagonist, and even more particularly, a CB1 inverse agonist. This invention relates to liquid compositions of Compound I and pharmaceutically acceptable salts and solvates thereof for use in mammals, especially humans, which compositions can be administered parenterally and which provide an increased concentration of Compound I for absorption.

Compound I is an inverse agonist of the Cannabinoid-1 (CB1) receptor, and compositions of the present invention comprising Compound I are useful in the treatment, prevention, and suppression of one or more diseases mediated by the Cannabinoid-1 (CB1) receptor, including psychosis; memory deficits; cognitive disorders; migraine; neuropathy; neuro-inflammatory disorders including multiple sclerosis and Guillain-Barre syndrome and the inflammatory sequelae of viral encephalitis, cerebral vascular accidents, and head trauma; anxiety disorders; stress; epilepsy; Parkinson's disease; movement disorders; schizophrenia; substance abuse disorders, particularly of opiates, alcohol, marijuana, and nicotine, including smoking cessation; obesity; eating disorders associated with excessive food intake and complications associated therewith; constipation; chronic intestinal pseudo-obstruction; cirrhosis of the liver; diabetes; and asthma.

The pharmaceutical industry is faced with the challenge of developing compositions for an increasing number of active molecules that possess low aqueous solubility and/or low intestinal epithelial permeability. Compound I possesses a very low aqueous solubility (<0.4 μg/mL) and thus acceptable bioavailability can not readily be achieved by means of traditional liquid and solid compositions after oral administration. Liquid-filled capsule dosage forms of Compound I for oral administration are described in WO 2006/057903. There remains a need to develop compositions of Compound I that would increase the solubility of Compound I and thus allow for effective administration of the compound at the required dose.

### SUMMARY OF THE INVENTION

A liquid pharmaceutical composition is described comprising N-[1S,2S]-3-[(4-chlorophenyl)-2-(3-cyanophenyl)-1-methylpropyl]-2-methyl-2-{[(5-trifluoromethyl)pyridine-2-yl]oxy}propanamide (Compound I) or a pharmaceutically acceptable salt or solvate thereof; water; one or more hydrophilic non-ionic surfactant(s) having a hydrophobic-lipophilic balance (HLB) of 8-20; one or more cosolvent(s); and a tonicity modifier.

Also disclosed is a process for preparing a liquid pharmaceutical composition, comprising mixing together N-[1S,2S]-3-[(4-chlorophenyl)-2-(3-cyanophenyl)-1-methylpropyl]-2-methyl-2-{[(5-trifluoromethyl)pyridine-2-yl]oxy}propanamide (Compound I) or a pharmaceutically acceptable salt or solvate thereof, water, one or more hydrophilic non-ionic surfactant(s) having a HLB of 8-20, one or more cosolvent(s) and a tonicity modifier.

An additional aspect of the invention is a process for preparing a vessel containing a liquid composition for parenteral administration comprising the steps of mixing Compound I or a pharmaceutically acceptable salt or solvate thereof and one or more cosolvent(s); adding a hydrophilic non-ionic surfactant(s) having a HLB of 8-20, and mixing; adding water, and mixing; adding a tonicity modifier, and mixing; filtering the composition through a PVDF membrane into a sterile vessel via a PTFE coated rubber tubing.

Another aspect of the invention is a method of treating or preventing diabetes or obesity, which method comprises administration to a patient in need thereof of an effective amount of a liquid pharmaceutical composition of any one of claims 1 to 5.

Yet another aspect of the invention is the use of a liquid pharmaceutical composition of any one of claims 1 to 5 for the manufacture of a medicament for the treatment or prevention of diabetes or obesity.

### DETAILED DESCRIPTION OF THE INVENTION

It has been found that the aqueous solubility of Compound I is increased by the presence of one or more hydrophilic non-ionic surfactants in the liquid compositions.

The present invention relates to a liquid pharmaceutical composition comprising:
(a) N-[1S,2S]-3-[(4-chlorophenyl)-2-(3-cyanophenyl)-1-methylpropyl]-2-methyl-2-{[(5-trifluoromethyl)pyridine-2-yl]oxy}propanamide (Compound I) or a pharmaceutically acceptable salt or solvate thereof;
(b) water;
(c) one or more hydrophilic non-ionic surfactant(s) having a hydrophobic-lipophilic balance (HLB) of 8-20;
(d) one or more cosolvent(s); and
(e) a tonicity modifier.

The ratio of Compound I, water, surfactant(s), cosolvent(s) and tonicity modifier depends upon the desired solubility and concentration, which in turn is dependent on the required dose per unit of Compound I.

In an embodiment one or more antioxidant(s) are present.

In an embodiment one or more antimicrobial(s) are present.

In an embodiment one or more antioxidant(s) and one or more antimicrobial(s) are present.

In an embodiment, Compound I is unsolvated. In another embodiment, Compound I is a solvate or a hemisolvate.

In another embodiment, Compound I is an unsolvated free base.

In another embodiment, Compound I is an unsolvated salt. In another embodiment, Compound I is a solvated salt.

The amount of Compound I, the active ingredient, present in the composition is dependent on the required dose per unit of Compound I and can accordingly be ascertained by a person skilled in the art. The dosage of Compound I administered to a patient will generally be known or determined by the attending physician. In general, an effective dose for Compound I is from 0.01 mg to about 1000 mg per day, in single or divided doses; preferably from about 0.1 mg to about 10 mg per day, in single or divided doses. For parenteral administration, the liquid compositions contain up to 0.08 mg/mL of the active ingredient to provide from 0.01 to 20 mg, preferably 0.01, 0.05, 0.1, 0.5, 1, 2, 2.5, 3, 4, 5, 6, 7, 7.5, 8, 9, 10, 15 or 20, most preferably 2, 4, or 6 mg of the active ingredient for the symptomatic adjustment of the dosage to the patient to be treated.

In an embodiment Compound I forms about 0.004% of the total composition.

The amount of water present in the composition is dependent on the desired concentration of Compound I and can accordingly be ascertained by persons skilled in the art. The desired concentration will depend on the required dosage per unit of Compound I.

In an embodiment water forms about 98% of the total composition.

In an embodiment, the hydrophilic non-ionic surfactant has an HLB of 10-20. In another embodiment the hydrophilic non-ionic surfactant is selected from: polyoxyethylene 20 sorbitan monooleate, Polysorbate 80; polyoxyethylene 20 sorbitan monolaurate, Polysorbate 20; polyethylene (40 or 60) hydrogenated castor oil; polyoxyethylene (35) castor oil; polyethylene (60) hydrogenated castor oil; alpha tocopheryl polyethylene glycol 1000 succinate; glyceryl PEG 8 caprylate/caprate (caprylocaproyl macrogol glycerides); PEG 32 glyceryl laurate (lauroyl macrogol glycerides); stearyl macrogol glycerides; polyoxyethylene fatty acid esters; polyoxyethylene fatty acid ethers; and Poloxamers (124, 188, 407). In another embodiment, the hydrophilic non-ionic surfactants include: Polysorbate 80, polyoxyl 40 hydrogenated castor oil, polyoxyl 35 castor oil and glyceryl PEG 8 caprylate/caprate. In another embodiment, the hydrophilic non-ionic surfactant is selected from: Polysorbate 80; polyethylene (40 or 60) hydrogenated castor oil; glyceryl PEG 8 caprylate/caprate; and alpha tocopheryl polyethylene glycol 1000 succinate. In another embodiment, the hydrophilic non-ionic surfactant is Polysorbate 80.

The amount of surfactant present in the composition is dependent on the desired solubility of Compound I and can accordingly be ascertained by persons skilled in the art. The desired solubility will depend on the required dosage per unit of Compound I.

In an embodiment one or more surfactant(s) form about 0.5% of the total composition.

The addition of one or more cosolvent(s) to a liquid pharmaceutical composition comprising Compound I can facilitate the manufacturing process.

In an embodiment, the cosolvent is an alcohol. In another embodiment the cosolvent is selected from: 1,2-propylene glycol (PG), ethanol, glycerol and polyethylene glycols. In another embodiment the cosolvent is ethanol.

The amount of cosolvent present in the composition is dependent on the desired concentration of Compound I and can accordingly be ascertained by persons skilled in the art. The desired concentration will depend on the required dosage per unit of Compound I.

In an embodiment one or more cosolvent(s) form about 0.5% of the total composition.

The tonicity modifiers in the liquid compositions of the present invention ensure that the tonicity, i.e., osmolality, of the solution is essentially the same as normal physiological fluids and thus prevent post-administration swelling or rapid absorption of the composition because of differential ion concentrations between the composition and physiological fluids. The tonicity modifier is present in a suitable amount to achieve an isotonic solution and the amount can be ascertained by persons skilled in the art with the aid of no more than routine experimentation. In an embodiment the amount of tonicity modifier used is up to about 5% (w/v). In another embodiment the tonicity modifier forms about 0.09% of the total formulation. The particular tonicity modifier used is not critical to the practice of the present invention. The preferred tonicity of the liquid formulation is about 280 to about 320 mmol/kg, particularly about 300 mmol/kg.

Examples of suitable tonicity modifiers include sodium chloride, glycerin, boric acid, calcium chloride, dextrose, and potassium chloride. In an embodiment the tonicity modifier is sodium chloride, glycerin or dextrose. In another embodiment the tonicity modifier is sodium chloride.

Examples of suitable antioxidants are a (a or alpha ?)-tocopherol and ascorbate.

Examples of suitable antimicrobials are parabens, chlorobutanol and phenol.

The HLB, an acronym for “hydrophobic-lipophilic balance”, is a rating scale which can range from 1-20 for non-ionic surfactants. The higher the HLB; the more hydrophilic the surfactant. Hydrophilic surfactants (HLB 8-20), when used alone, provide fine emulsions which are, advantageously, more likely to empty uniformly from the stomach and provide a much higher surface area for absorption. Hydrophilic surfactants having an HLB of 8-20 include surfactants having HLB of 8, 9, 10, 11, 12, 13, 14, 15, 16, 17, 18, 19 and 20.

The term “pharmaceutically acceptable salts” refers to salts prepared from pharmaceutically acceptable acids, including inorganic or organic acids. Examples of pharmaceutically acceptable salts further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations.

Reference to a specific weight or percentage of “active ingredient”, Compound I, or N-[1S,2S]-3-(4-chlorophenyl)-2-(3-cyanophenyl)-1-methylpropyl]-2-methyl-2-{[5-trifluoromethyl]pyridine-2-yl}oxy}propanamide, is on the basis of the free base weight, absent the weight of any counterion or solvate present, unless otherwise indicated. For example, the phrase “1 mg N-[1S,2S]-3-(4-chlorophenyl)-2-(3-cyanophenyl)-1-methylpropyl]-2-methyl-2-{[5-trifluoromethyl]pyridine-2-yl}oxy}propanamide MTBE hemisolvate” means that the amount of the compound selected is based on 1 mg of N-[1S,2S]-3-(4-chlorophenyl)-2-(3-cyanophenyl)-1-methylpropyl]-2-methyl-2-{[5-trifluoromethyl]pyridine-2-yl}oxy}propanamide as the free base, absent the weight of the solvent present in the solvate.

The liquid compositions of the present invention are particularly suitable for parenteral administration into a subject. The subject is a mammal, such as a rodent (e.g. a guinea pig, a hamster, a rat, a mouse), murine (e.g. a mouse), feline (e.g. a cat), equine (e.g. a horse) or a primate such as simian (e.g. a monkey or ape), a monkey (e.g. marmoset, baboon), an ape (e.g. gorilla, chimpanzee, orangutan, gibbon), or a human. The compositions of this invention are particularly useful for administering to humans.

The expression “parenteral administration” as used herein refers to routes of administration other than through the gastrointestinal tract or lungs. Thus, “parenteral” as used herein includes, for example, intramuscular, subcutaneous, intra-articular (i.e. into the joint, which in turn includes intra-synovial, i.e. into the synovial fluid) and, especially, intravenous routes. The words “parenteral” and “injectable” can be used interchangeably. The words ‘compositions’ and ‘formulations’ can be used interchangeably. The liquid compositions of the present invention are particularly suitable for intravenous administration.

The present invention also provides a liquid pharmaceutical composition comprising:
(a) Compound I, or a pharmaceutically acceptable salt or solvate thereof;
(b) water;
(c) one or more hydrophilic non-ionic surfactant(s) selected from polysorbate 80, polyoxyl 40 hydrogenated castor oil, polyoxyl 35 castor oil and glyceryl PEG 8 caprylate/caprate;
(d) one or more cosolvent(s) selected from 1,2-propylene glycol (PG), ethanol, glycerol and polyethylene glycols;
(e) a tonicity modifier selected from sodium chloride, glycerin, boric acid, calcium chloride, dextrose, and potassium chloride.

The present invention also provides a liquid pharmaceutical composition comprising:
(a) Compound I, or a pharmaceutically acceptable salt or solvate thereof,
(b) water;
(c) polysorbate 80;
(d) a cosolvent selected from propylene glycol and ethanol;
(e) a tonicity modifier selected from sodium chloride, glycerin, boric acid, calcium chloride, dextrose, and potassium chloride.

The present invention also provides a liquid pharmaceutical composition comprising:
(a) Compound I, or a pharmaceutically acceptable salt or solvate thereof;
(b) water;
(c) polysorbate 80;
(d) ethanol;
(e) sodium chloride.

In an embodiment is provided a liquid pharmaceutical composition comprising:
(a) about 0.004% of Compound I, or a pharmaceutically acceptable salt or solvate thereof;
(b) about 98% of water;
(c) about 0.5% of one or more hydrophilic non-ionic surfactant(s) having a HLB of 8-20;
(d) about 0.5% of one or more cosolvent(s); and
(e) about 0.9% of a tonicity modifier.

In an embodiment is provided a liquid pharmaceutical composition comprising:
(a) about 0.004% of Compound I, or a pharmaceutically acceptable salt or solvate thereof;
(b) about 98% of water;
(c) about 0.5% of polysorbate 80;
(d) about 0.5% of ethanol;
(e) about 0.9% of sodium chloride.

The present invention also provides a vessel comprising any one of the liquid pharmaceutical compositions defined above.

The present invention also provides a process for preparing a liquid pharmaceutical composition, comprising mixing together N-[1S,2S]-3-[(4-chlorophenyl)-2-(3-cyanophenyl)-1-methylpropyl]-2-methyl-2-{[(5-trifluoromethyl)pyridine-2-yl]oxy}propanamide (Compound I) or a pharmaceutically acceptable salt or solvate thereof, water, one or more hydrophilic non-ionic surfactant(s) having a HLB of 8-20, one or more cosolvent(s) and a tonicity modifier.

The present invention also provides a process for preparing a liquid pharmaceutical composition comprising the steps of:
(a) mixing Compound I or a pharmaceutically acceptable salt or solvate thereof and one or more cosolvent(s);
(b) adding one or more hydrophilic non-ionic surfactant(s) having a HLB of 8-20, and mixing;
(c) adding water, and mixing; and
(d) adding a tonicity modifier, and mixing.

The present invention also provides a process for preparing a liquid pharmaceutical composition for parenteral administration comprising the steps of:
(a) mixing Compound I or a pharmaceutically acceptable salt or solvate thereof and ethanol;
(b) adding polysorbate 80, and mixing;
(c) adding water, and mixing; and
(d) adding sodium chloride, and mixing.

In an embodiment of each of the above process embodiments, one or more of the hydrophilic non-ionic surfactant(s) are also diluted with water before adding to the composition.

In an embodiment of each of the above embodiments, the liquid composition is filtered, for example through a polyvinylidene Difluoride (PVDF) membrane.

In an embodiment of each of the above process embodiments, the liquid composition is filtered, for example through a PVDF membrane, into a sterile vessel.

In an embodiment of each of the above process embodiments, the composition is filtered, for example through a PVDF membrane, into a sterile vessel, for example via a polytetrafluoroethylene (PTFE) coated rubber tubing.

The present invention also provides a process for preparing a vessel containing a liquid composition for parenteral administration comprising the steps of:
(a) mixing Compound I or a pharmaceutically acceptable salt or solvate thereof and one or more cosolvent(s);
(b) adding a hydrophilic non-ionic surfactant(s) having a HLB of 8-20, and mixing;
(c) adding water, and mixing;
(d) adding a tonicity modifier, and mixing;
(e) filtering the composition through a PVDF membrane into a sterile vessel via a PTFE coated rubber tubing;

In an embodiment the PVDF membrane is sterilized.

In an embodiment of any one of the processes above, the process is carried out in the order of the steps specified.

The composition of the present invention comprising Compound I are useful in the treatment, prevention and suppression of diseases mediated by the Cannabinoid-1 (CB1) receptor, including the diseases specified in WO 03/077847 and WO 2006/057903. Examples of such diseases are psychosis; memory deficits; cognitive disorders; migraine; neuropathy; neuro-inflammatory disorders including multiple sclerosis and Guillain-Barre syndrome and the inflammatory sequelae of viral encephalitis, cerebral vascular accidents, and head trauma; anxiety disorders; stress; epilepsy; Parkinson's disease; movement disorders; schizophrenia; substance abuse disorders, particularly to opiates, alcohol, marijuana, and nicotine, including smoking cessation; obesity; eating disorders associated with excessive food intake and complications associated therewith; constipation; chronic intestinal pseudo-obstruction; cirrhosis of the liver; diabetes; and asthma.

For example, the composition of the present invention is for use in treating obesity.

The composition of the present invention is for use in treating substance abuse disorders. In an embodiment, the substance abuse disorders are selected from abuse of opiates, alcohol, marijuana, and nicotine.

The composition of the present invention is useful for smoking cessation.

The composition of the present invention is for use in treating alcohol addiction.

The composition of the present invention is for use in treating a patient with diabetes.

In still another embodiment of the present invention, the pharmaceutical composition is for use in treating a patient who smokes. In one class of this embodiment, the patient no longer wishes to continue smoking.

The composition of the present invention is for use in treating a patient who is abusing a substance selected from opiates, alcohol, and marijuana.

The composition of the present invention is for use in treating a patient who is an alcoholic.

The terms “administration of” and or “administering a” compound should be understood to mean providing the composition of the invention to the subject in need of treatment.

The administration of the compositions of the present invention to practice the present methods of therapy is carried out by administering an effective amount of the compound of structural formula Ito the patient in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

The present invention also provides a combination of a liquid pharmaceutical composition as defined above and a nicotinic receptor partial agonist such as varenicline or SR 591813; or an antidepressant such as bupropion, doxepine, or nortriptyline; or an anxiolytic agent such as buspirone or clonidine, for simultaneous, separate or sequential administration.

For example, a nicotinic receptor partial agonist such as varenicline or SR 591813; or an antidepressant such as bupropion, doxepine, or nortriptyline; or an anxiolytic agent such as buspirone or clonidine can also be added to the compositions of the present invention for the treatment of substance abuse disorders.

Specific compounds of use in combination with Compound I of the present invention include: simvastatin, mevastatin, ezetimibe, atorvastatin, sitagliptin, metformin, sibutramine, orlistat, pioglitazone, rosiglitazone, Qnexa, topiramate, naltrexone, bupriopion, phentermine, losartan, and losartan with hydrochlorothiazide. Specific CB1 antagonists/inverse agonists of use in combination with Compound I of the present invention include those described in WO05/000809, which includes the following: 3-{1-[bis(4-chlorophenyl)methyl]azetidin-3-ylidene}-3-(3,5-difluorophenyl)-2,2-dimethylpropanenitrile, 1-{1-[1-(4-chlorophenyl)pentyl]azetidin-3-yl}-1-(3,5-difluorophenyl)-2-methylpropan-2-ol. 3-((S)-(4-chlorophenyl) {3-[(1S)-1-(3,5-difluorophenyl)-2-hydroxy-2-methylpropyl]azetidin-1-yl}methyl)benzonitrile, 3-((S)-(4-chlorophenyl){3-[(1S)-1-(3,5-difluorophenyl)-2-fluoro-2-methylpropyl]azetidin-1-yl}methyl)benzonitrile, 3-((4-chlorophenyl){3-[1-(3,5-difluorophenyl)-2,2-dimethylpropyl]azetidin-1-yl}methyl)benzonitrile, 3-((1S)-1-{1-[(S)-(3-cyanophenyl)(4-cyanophenyl)methyl]azetidin-3-yl}-2-fluoro-2-methylpropyl)-5-fluorobenzonitrile, 3-[(S)-(4-chlorophenyl)(3-{(1S)-2-fluoro-1-[3-fluoro-5-(4H-1,2,4-triazol-4-yl)phenyl]-2-methylpropyl}azetidin-1-yl)methyl]benzonitrile, and 5-((4-chlorophenyl){3-[(1S)-1-(3,5-difluorophenyl)-2-fluoro-2-methylpropyl]azetidin-1-yl}methyl)thiophene-3-carbonitrile, and pharmaceutically acceptable salts thereof; as well as: 3-[(S)-(4-chlorophenyl)(3-{(1S)-2-fluoro-1-[3-fluoro-5-(5-oxo-4,5-dihydro-1,3,4-oxadiazol-2-yl)phenyl]-2-methylpropyl}azetidin-1-yl)methyl]benzonitrile, 3-[(S)-(4-chlorophenyl)(3-{(1S)-2-fluoro-1-[3-fluoro-5-(1,3,4-oxadiazol-2-yl)phenyl]-2-methylpropyl}azetidin-1-yl)methyl]benzonitrile, 3-[(S)-(3-{(1S)-1-[3-(5-amino-1,3,4-oxadiazol-2-yl)-5-fluorophenyl]-2-fluoro-2-methylpropyl}azetidin-1-yl)(4-chlorophenyl)methyl]benzonitrile, 3-[(S)-(4-cyanophenyl)(3-{(1S)-2-fluoro-1-[3-fluoro-5-(5-oxo-4,5-dihydro-1,3,4-oxadiazol-2-yl)phenyl]-2-methylpropyl}azetidin-1-yl)methyl]benzonitrile, 3-[(S)-(3-{(1S)-1-[3-(5-amino-1,3,4-oxadiazol-2-yl)-5-fluorophenyl]-2-fluoro-2-methylpropyl}azetidin-1-yl)(4-cyanophenyl)methyl]benzonitrile, 3-[(S)-(4-cyanophenyl)(3-{(1S)-2-fluoro-1-[3-fluoro-5-(1,3,4-oxadiazol-2-yl)phenyl]-2-methylpropyl}azetidin-1-yl)methyl]benzonitrile, 3-[(S)-(4-chlorophenyl)(3-{(1S)-2-fluoro-1-[3-fluoro-5-(1,2,4-oxadiazol-3-yl)phenyl]-2-methylpropyl}azetidin-1-yl)methyl]benzonitrile, 3-[(1S)-1-(1-{(S)-(4-cyanophenyl)[3-(1,2,4-oxadiazol-3-yl)phenyl]-methyl}azetidin-3-yl)-2-fluoro-2-methylpropyl]-5-fluorobenzonitrile, 5-(3-{1-[1-(diphenylmethyl)azetidin-3-yl]-2-fluoro-2-methylpropyl}-5-fluorophenyl)-1H-tetrazole, 5-(3-{1-[1-(diphenylmethyl)azetidin-3-yl]-2-fluoro-2-methylpropyl}-5-fluorophenyl)-1-methyl-1H-tetrazole, 5-(3-{1-[1-(diphenylmethyl)azetidin-3-yl]-2-fluoro-2-methylpropyl}-5-fluorophenyl)-2-methyl-2H-tetrazole, 3-[(4-chlorophenyl)(3-{2-fluoro-1-[3-fluoro-5-(2-methyl-2H-tetrazol-5-yl)phenyl]-2-methylpropyl}azetidin-1-yl)methyl]benzonitrile, 3-[(4-chlorophenyl)(3-{2-fluoro-1-[3-fluoro-5-(1-methyl-1H-tetrazol-5-yl)phenyl]-2-methylpropyl}azetidin-1-yl)methyl]benzonitrile, 3-[(4-cyanophenyl)(3-{2-fluoro-1-[3-fluoro-5-(1-methyl-1H-tetrazol-5-yl)phenyl]-2-methylpropyl}azetidin-1-yl)methyl]benzonitrile, 3-[(4-cyanophenyl)(3-{2-fluoro-1-[3-fluoro-5-(2-methyl-2H-tetrazol-5-yl)phenyl]-2-methylpropyl}azetidin-1-yl)methyl]benzonitrile, 5-{3-[(S)-{3-[(1S)-1-(3-bromo-5-fluorophenyl)-2-fluoro-2-methylpropyl]azetidin-1-yl}(4-chlorophenyl)methyl]phenyl}-1,3,4-oxadiazol-2(3H)-one, 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-cyanophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-cyanophenyl)[3-(1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-chlorophenyl) [3-(1,3,4-oxadiazol-2-yl)phenyl]methyl}azetidin-3-yl)-2-fluoro-2-methylpropyl]-5-fluorobenzonitrile, 3-((1S)-1-{1-[(S)-[3-(5-amino-1,3,4-oxadiazol-2-yl)phenyl](4-chlorophenyl)methyl]azetidin-3-yl}-2-fluoro-2-methylpropyl)-5-fluorobenzonitrile, 3-((1S)-1-{1-[(S)-[3-(5-amino-1,3,4-oxadiazol-2-yl)phenyl](4-cyanophenyl)methyl]azetidin-3-yl}-2-fluoro-2-methylpropyl)-5-fluorobenzonitrile, 3-[(1S)-1-(1-{(S)-(4-cyanophenyl) [3-(1,2,4-oxadiazol-3-yl)phenyl]methyl}azetidin-3-yl)-2-fluoro-2-methylpropyl]-5-fluorobenzonitrile, 3-[(1S)-1-(1-{(S)-(4-chlorophenyl)[3-(1,2,4-oxadiazol-3-yl)phenyl]methyl}azetidin-3-yl)-2-fluoro-2-methylpropyl]-5-fluorobenzonitrile, 5-[3-((S)-(4-chlorophenyl) {3-[(1S)-1-(3,5-difluorophenyl)-2-fluoro-2-methylpropyl]azetidin-1-yl}methyl)phenyl]-1,3,4-oxadiazol-2(3H)-one, 5-[3-((S)-(4-chlorophenyl){3-[(1S)-1-(3,5-difluorophenyl)-2-fluoro-2-methylpropyl]azetidin-1-yl}methyl)phenyl]-1,3,4-oxadiazol-2(3H)-one, 4-{(S)-{3-[(1S)-1-(3,5-difluorophenyl)-2-fluoro-2-methylpropyl]azetidin-1-yl}[3-(5-oxo-4,5-dihydro-1,3,4-oxadiazol-2-yl)phenyl]methyl}-benzonitrile, and pharmaceutically acceptable salts thereof.

Specific NPY5 antagonists of use in combination with a compound of the present invention include: 3-oxo-N-(5-phenyl-2-pyrazinyl)-spiro[isobenzofuran-1(3H), 4′-piperidine]-1′-carboxamide trans-N-[1-(2-fluorophenyl)-3-pyrazolyl]-3-oxo spiro[6-azaisobenzofuran-1(3H), 1′-cyclohexane]-4′-carboxamide, and pharmaceutically acceptable salts and esters thereof.

Specific ACC-1/2 inhibitors of use in combination with Compound I include: 1′-[(4,8-dimethoxyquinolin-2-yl)carbonyl]-6-(1H-tetrazol-5-yl)spiro[chroman-2,4′-piperidin]-4-one, (5-{1′-[(4,8-dimethoxyquinolin-2-yl)carbonyl]-4-oxospiro[chroman-2,4′-piperidin]-6-yl}-2H-tetrazol-2-yl)methyl pivalate, 5-{1′-[(8-cyclopropyl-4-methoxyquinolin-2-yl)carbonyl]-4-oxospiro[chroman-2,4′-piperidin]-6-yl}nicotinic acid, 1′-(8-methoxy-4-morpholin-4-yl-2-naphthoyl)-6-(1H-tetrazol-5-yl)spiro[chroman-2,4′-piperidin]-4-one, and l′-[(4-ethoxy-8-ethylquinolin-2-yl)carbonyl]-6-(1H-tetrazol-5-yl)spiro[chroman-2,4′-piperidin]-4-one, and pharmaceutically acceptable salts thereof.

Specific MCH1R antagonist compounds of use in combination with Compound I include: 1-{-4-[(1-ethylazetidin-3-yl)oxy]phenyl}-4-[(4-fluorobenzyl)oxy]pyridin-2(1H)-one, 4-[(4-fluorobenzyl)oxy]-1-{4-[(1-isopropylazetidin-3-yl)oxy]phenyl}pyridin-2(1H)-one, 1-[4-(azetidin-3-yloxy)phenyl]-4-[(5-chloropyridin-2-yl)methoxy]pyridin-2(1H)-one, 4-[(5-chloropyridin-2-yl)methoxy]-1-{4-[(1-ethylazetidin-3-yl)oxy]phenyl}pyridin-2(1H)-one, 4-[(5-chloropyridin-2-yl)methoxy]-1-{4-[(1-propylazetidin-3-yl)oxy]phenyl}pyridin-2(1H)-one, and 4-[(5-chloropyridin-2-yl)methoxy]-1-(4-{[(2S)-1-ethylazetidin-2-yl]methoxy}phenyl)pyridin-2(1H)-one, or a pharmaceutically acceptable salt thereof.

Specific DP-IV inhibitors of use in combination with a compound of the present invention are selected from 7-[(3R)-3-amino-4-(2,4,5-trifluorophenyl)butanoyl]-3-(trifluoromethyl)-5,6,7,8-tetrahydro-1,2,4-triazolo[4,3-a]pyrazine. In particular, Compound I is favorably combined with 7-[(3R)-3-amino-4-(2,4,5-trifluorophenyl)butanoyl]-3-(trifluoromethyl)-5,6,7,8-tetrahydro-1,2,4-triazolo[4,3-a]pyrazine, and pharmaceutically acceptable salts thereof.

Further agents which can be used in combination with the liquid pharmaceutical composition of the present invention are described in WO 03/077847.

The surfactants which can be used in the present invention are available commercially under the following trademarks:

Polysorbate 80 (TWEEN 80 from ICI, CRILLET 4 NF and CRILLET 4 HP from Croda and NOFABLE ESO-9920 from NOF Corporation); polysorbate 20 (TWEEN 20 from ICI and CRILLET 1 NF and CRILLET 1 HP from Croda); polyethylene (40 or 60) hydrogenated castor oil (CREMOPHOR EL/RH40 and RH60 from BASF); polyoxyethylene (35) castor oil (CREMOPHOR EL from BASF and ETOCAS 30 from Croda)); polyethylene (60) hydrogenated castor oil (NIKKOL HCO-60); alpha tocopheryl polyethylene glycol 1000 succinate (Vitamin E TPGS from Eastman); glyceryl PEG 8 caprylate/caprate (caprylocaproyl macrogol glycerides available as LABRASOL from Gattefosse, and under the tradename ACCONON MC-8 from Abitec Corp.); PEG 32 glyceryl laurate (lauroyl macrogol glycerides sold commercially under the registered trademark GELUCIRE 44/14 from Gattefosse); stearyl macrogol glycerides (GELUCIRE 50/13 from Gattefosse); polyoxyethylene fatty acid esters (MYRJ from ICI); polyoxyethylene fatty acid ethers (BRIJ from ICI); and Poloxamers (124, 188, 407) (LUTROLS or PLURONICS from BASF).

Examples of methods of synthesis of Compound I are described in WO 03/077847 and WO 2006/057903. Other synthesis methods may also be used.

Abbreviations: DMF: dimethylformamide; ee: enantiomeric excess; HLB: hydrophilic-lipophilic balance; in: inches; LCAP: liquid chromatography assay percent; Me: methyl; MTBE: methyl tert-butyl ether; PEG: polyethylene glycol; PG: propylene glycol; RT: room temperature; SOLKA FLOC: filter aid.

### PREPARATORY EXAMPLE 1

### N-[1S,2S]-3-(4-chlorophenyl)-2-(3-cyanophenyl)-1-methylpropyl]-2-methyl-2-{[5-(trifluoromethylpyridin-2-yl)oxy]propanamide MTBE hemisolvate

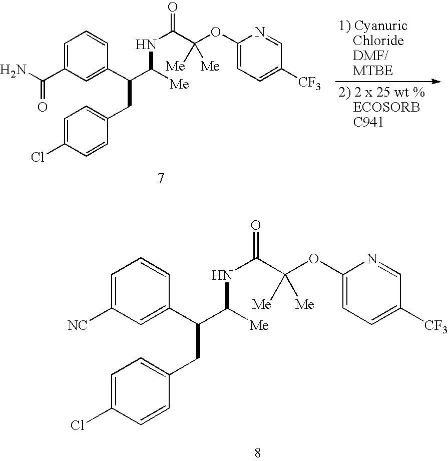

A solution of 470 g of 3-{(1S,2S)-1-(4-chlorobenzyl)-2-[(2-methyl-2-{[5-(trifluoromethyl)pyridine-2-yl]oxy}propanoyl)amino]-propyl}benzamide in DMF is transferred to a 12 L 4-necked round bottom flask equipped with mechanical stirrer, thermocouple, and 2 L addition funnel. Cyanuric chloride (103 g) is slurried in 2 L of MTBE and the resulting slurry was charged to the reaction via the 2 L addition funnel over ˜10 minutes. The reaction mixture is aged with stirring for 1 hour. The batch is cooled to 10° C. and diluted with 3 L of MTBE. 2 L of water and 2 L of saturated NaHCO₃ solution are added to the reaction while keeping the temperature below 20° C. The resulting slurry is transferred to a 50 L extractor containing 3 L of MTBE, 3 L of water, and 3 L of sat'd NaHCO₃. An additional 12 L of water is added to the batch and the layers are allowed to settle. The organic layer is washed twice with 3 L of water. Ecosorb Treatment/Hemisolvate Isolation: The organic layer is azeotroped at 35° C., 17 in Hg to bring the KF to 219 (spec. at 500) while maintaining a volume of ˜11 L. The batch is then treated with 320 g of ECOSORB C941. The batch is aged for 4 hours at 50° C., then filtered over a pad of SOLKA FLOC and washed with 6 L of MTBE. The resulting filtrate is recharged to a 22 L vessel, concentrated to 11 L volume, and retreated with 116 g of ECOSORB C941. This slurry is filtered over a bed of SOLKA FLOC, and washed with 6 L MTBE. The resulting colorless MTBE layer is transferred through a 1 micron inline filter into a 12 L, 4 neck round bottom flask equipped with overhead stirrer and thermocouple, and concentrated to ˜2 L volume at 17 in Hg, 35° C. The batch is cooled to RT, and a sample is removed to create a seed bed. Once the sample is crystallized, it is returned to the flask, and the batch is aged for 30 minutes, creating a large seed bed. The isolated solid is dried over a stream of nitrogen to afford the title compound as an MTBE hemisolvate.

### PREPARATORY EXAMPLE 2

Isolation of N-[1S,2S]-3-(4-chlorophenyl)-2-(3-cyanophenyl)-1-methylpropyl]-2-methyl-2-{[5-(trifluoromethyl pyridin-2-yl)oxy]propanamide Polymorph B

In a 3 L, 3 neck round bottom flask equipped with overhead stirrer and thermocouple, 350 g of N-[1S,2S]-3-(4-chlorophenyl)-2-(3-cyanophenyl)-1-methylpropyl]-2-methyl-2-{[5-(trifluoromethylpyridin-2-yl)oxy]propanamide hemisolvate was slurried in a total of 1.82 L of 2:3 isopropyl acetate:heptane. The mixture was aged for 1 h, and then filtered over a very small bed of SOLKA FLOC, thoroughly pull the liquors from the filter bed to minimize the loss of mother liquors. The filter cake was washed with 1 L of 1:3 IPAc: heptane into a separate flask. The two filtrates were combined (combined ee=98.5% ee). These two solutions were transferred by vacuum through a 1 micron inline filter into a 22 L 4 neck round bottom flask. The batch was heated to 45° C. over a steam pot, and then charged with 2.35 L of heptane. Seed of N-[1S,2S]-3-(4-chlorophenyl)-2-(3-cyanophenyl)-1-methylpropyl]-2-methyl-2-{[5-(trifluoromethyl pyridin-2-yl)oxy]propanamide Polymorph B (Polymorph B seed was obtained from the same solvent system over a long time frame) (15.0 g) was added and the batch was aged at 45° C. overnight. The resulting slurry was then charged with 150 mL of heptane over 5 hours, then 220 mL heptane at 2.0 mL/min, then 1131 mL of heptane at 9 mL/min, then 6783 mL of heptane at 60 mL/min. Once all heptane was charged, the batch was cooled to RT and aged overnight. The batch was cooled to 0° C. and aged for 1 hour, filtered, and washed with 1 L of heptane to afford the title compound, crystal Form B (287 g, 87% isolated yield (from hemisolvate and corrected for seed), 98.6% ee, 99.5 LCAP, 99.5 wt % assay).

### Example 1

### Solubility of N-[1S,2S]-3-(4-chlorophenyl)-2-(3-cyanophenyl)-1-methylpropyl]-2-methyl-2-{[5-trifluoromethyl]pyridine-2-yl}oxy}propanamide anhydrous, Polymorph B in Various Liquid Vehicles

Solubility determinations were carried out at room temperature unless otherwise specified. Solubility of N-[1S,2S]-3-(4-chlorophenyl)-2-(3-cyanophenyl)-1-methylpropyl]-2-methyl-2-{[5-trifluoromethyl]pyridine-2-yl}oxy}propanamide (Compound I) as anhydrous unsolvated Polymorph B (such as prepared in Preparatory Example 2) was determined by preparing a suspension of anhydrous Polymorph B of Compound I in the solvent system. After equilibration for at least 24 hours, the suspension was filtered and the supernatant was analyzed by HPLC. Chromatography was performed on either a Vydac C₁₈ 300 Å 250×4.6 mm 5 μm particle size with in-line Phenomenex Security Guard w/C₁₈ cartridge or on a Polaris C₁₈-Ether columns or on a Polaris C₈-Ether columns using 0.1% phosphoric acid in combination with methanol or acetonitrile depending on HPLC conditions. The compound was detected at 220 and 272 nm wavelength and assayed using standard curves. The results are presented below in Table 1, calculated based on mg/g vehicle.

TABLE 1

| | Solubility of |
|---|---|
| Solvent | Compound I, mg/mL |
| 10% glycerol, 90% H₂0 | <0.001 |
| 10% EtOH, 90% H₂O | <0.001 |
| 15% glycerol, 10% EtOH, 75% H₂O | <0.001 |
| 25% PEG400, 75% H₂O | 0.004 |
| 35% Captisol, 15% glycerol, | 0.02 |
| 10% EtOH, 40% H₂0 | |
| 35% hydroxypropyl beta cyclodextrin, | 0.04 |
| 18% glycerol, 10% EtOH, 37% H₂0 | |
| 0.25% Tween80, 99.75% H₂0 | 0.04 |
| 25% PG, 25% PEG300, 50% H₂0 | 0.07 |
| 0.5% Tween80, 95.5% H₂0 | 0.08 |
| 0.5% Tween80, 5% EtOH, 94.5% H₂0 | 0.08 |
| 0.5% Tween80, 95.5% Saline | 0.08 |
| 2.5% Tween80, 97.5% H₂0 | 0.4 |

### Example 2

An example of the procedure used to prepare vials containing parenteral dosage forms of Compound I is given below:
1) Weigh the empty stainless steel formulation vessel (316 stainless steel) and record weight.
2) To this add 236.5 mg of Compound I bulk drug (endotoxin tested as per USP) that has been pre-weighed on an analytical balance (since the weight will be below the limit of measurement for the process balance).
3) To the bulk drug add 29.57 mL of Ethanol and agitate to dissolve the bulk drug and record weight.
4) To the above add polysorbate 80 solution and record weight. The contents are mixed with an overhead lightning mixer for 10 minutes. Polysorbate 80 solution is prepared by weighing 29.57 g of polysorbate 80 (endotoxin tested as per USP) into a 500 mL glass beaker on a top loading balance and solubilizing in approximately 250 mL of water for injections.
5) Add water for injection equivalent to 90% of final weight to the formulation vessel and record weight. The contents are mixed using a lightning mixer for approximately 15-30 minutes till a clear solution is obtained.
6) Add 53.22 g of Sodium chloride (endotoxin tested as per USP) to the above solution, record weight, and mix the contents using a lightning mixer till a clear solution results.
7) Make up the batch weight (calculated using a density of 1.0024 mg/mL) with water for injection and stir the final formulation for another 15 minutes to homogenize the contents. Measure and record the pH of the final formulation (target pH=5.2 with range from about 4.5 to 6.0).
8) Seal the formulation vessel (inlet and outlet ports) and transfer to a class 100 area.
9) In the class 100 area, filter the formulation through a pre-sterilized 0.22 μm PVDF membrane filter (Millipore; tested pre- and post-filtration for filter integrity) using a PTFE coated (inner surface of the tubing) rubber tubing into a sterile vessel.
10) Calibrate the peristaltic pump to fill the expected weight into the receiving vials.
11) Fill the target fill weight from the sterile filtered formulation vessel into sterilized clear Type I molded glass vials using a peristaltic pump. Stopper the glass vials with PTFE coated rubber stoppers (pre-sterilized by autoclaving).
12) Cap the vials and label them.

While the invention has been described and illustrated with reference to certain particular embodiments thereof, those skilled in the art will appreciate that various changes, modifications and substitutions can be made therein without departing from the spirit and scope of the invention. It is intended, therefore, that the invention be defined by the scope of the claims which follow and that such claims be interpreted as broadly as is reasonable.

1. A liquid pharmaceutical composition comprising:
(a) N-[1S,2S]-3-[(4-chlorophenyl)-2-(3-cyanophenyl)-1-methylpropyl]-2-methyl-2-{[(5-trifluoromethyl)pyridine-2-yl]oxy}propanamide (Compound I) or a pharmaceutically acceptable salt or solvate thereof;
(b) water;
(c) one or more hydrophilic non-ionic surfactant(s) having a hydrophobic-lipophilic balance (HLB) of 8-20;
(d) one or more cosolvent(s); and
(e) a tonicity modifier.

2. The liquid pharmaceutical composition of claim 1 wherein the hydrophilic non-ionic surfactant is selected from nonionic surfactants, such as polyoxyethylene 20 sorbitan monooleate, Polysorbate 80; polyoxyethylene 20 sorbitan monolaurate, Polysorbate 20; polyethylene (40 or 60) hydrogenated castor oil; polyoxyethylene (35) castor oil; polyethylene (60) hydrogenated castor oil; alpha tocopheryl polyethylene glycol 1000 succinate; glyceryl PEG 8 caprylate/caprate (caprylocaproyl macrogol glycerides); PEG 32 glyceryl laurate (lauroyl macrogol glycerides); stearyl macrogol glycerides; polyoxyethylene fatty acid esters; polyoxyethylene fatty acid ethers; and Poloxamers (124, 188, 407).

3. The liquid pharmaceutical composition of claim 1 comprising:
(a) Compound I, or a pharmaceutically acceptable salt or solvate thereof;
(b) water;
(c) one or more hydrophilic non-ionic surfactant(s) selected from polysorbate 80, polyoxyl 40 hydrogenated castor oil, polyoxyl 35 castor oil and glyceryl PEG 8 caprylate/caprate;
(d) one or more cosolvent(s) selected from 1,2-propylene glycol (PG), ethanol, glycerol and polyethylene glycols; and
(e) a tonicity modifier selected from sodium chloride, glycerin, boric acid, calcium chloride, dextrose, and potassium chloride.

4. The liquid pharmaceutical composition of claim 1 comprising:
(a) Compound I, or a pharmaceutically acceptable salt or solvate thereof;
(b) water;
(c) polysorbate 80;
(d) a cosolvent selected from propylene glycol and ethanol;
(e) a tonicity modifier selected from sodium chloride, glycerin, boric acid, calcium chloride, dextrose, and potassium chloride.

5. The liquid pharmaceutical composition of claim 1 comprising:
(a) about 0.004% of Compound I, or a pharmaceutically acceptable salt or solvate thereof;
(b) about 98% of water;
(c) about 0.5% of one or more hydrophilic non-ionic surfactant(s) having a HLB of 8-20;
(d) about 0.5% of one or more cosolvent(s); and
(e) about 0.9% of a tonicity modifier.

6. A vessel comprising a liquid pharmaceutical composition of any previous claim.

7. A process for preparing a liquid pharmaceutical composition, comprising mixing together N-[1S,2S]-3-(4-chlorophenyl)-2-(3-cyanophenyl)-1-methylpropyl]-2-methyl-2-{[(5-trifluoromethyl)pyridine-2-yl]oxy}propanamide (Compound I) or a pharmaceutically acceptable salt or solvate thereof, water, one or more hydrophilic non-ionic surfactant(s) having a HLB of 8-20, one or more cosolvent(s) and a tonicity modifier.

8. The process of claim 7 comprising the steps of
(a) mixing Compound I or a pharmaceutically acceptable salt or solvate thereof and one or more cosolvent(s);
(b) adding one or more hydrophilic non-ionic surfactant(s) having a HLB of 8-20, and mixing;
(c) adding water, and mixing; and
(d) adding a tonicity modifier, and mixing.

9. A process for preparing a vessel containing a liquid composition for parenteral administration comprising the steps of:
(a) mixing Compound I or a pharmaceutically acceptable salt or solvate thereof and one or more cosolvent(s);
(b) adding a hydrophilic non-ionic surfactant(s) having a HLB of 8-20, and mixing;
(c) adding water, and mixing;
(d) adding a tonicity modifier, and mixing;
(e) filtering the composition through a PVDF membrane into a sterile vessel via a PTFE coated rubber tubing.

10. A method of treating or preventing diabetes or obesity, which method comprises administration to a patient in need thereof of an effective amount of a liquid pharmaceutical composition of claim 1.

11. (canceled)
